SEQUENCE LISTING (22 sequences)

SEQ ID NO: 1 - DNA, 27 bp, Artificial
  probe
aaagtttccgtgtttcgttttgttcag

SEQ ID NO: 2 - DNA, 30 bp, Artificial
  probe
ctgttcatcaatataagtttccgtgtttcg

SEQ ID NO: 3 - DNA, 30 bp, Artificial
  probe
acggcaaatttgaagaggttttaactacat

SEQ ID NO: 4 - DNA, 30 bp, Artificial
  probe
ctgtagtgattaaataaaaaatacttcaga

SEQ ID NO: 5 - DNA, 30 bp, Artificial
  probe
cttctcagtatgtttgttcttctcagtatg

SEQ ID NO: 6 - DNA, 25 bp, Artificial
  probe
acttctcagtatgtttgttcttctc

SEQ ID NO: 7 - DNA, 30 bp, Artificial
  probe
catagataatttattatttatgacacaagt

SEQ ID NO: 8 - DNA, 30 bp, Artificial
  probe
tggatgtatcatcgctgatacggaaaatca

SEQ ID NO: 9 - DNA, 23 bp, Artificial
  probe
attgttggtgtgctgcgtgatcc

SEQ ID NO: 10 - DNA, 30 bp, Artificial
  probe
gaagtaagactgaatgatctctttcactgg

SEQ ID NO: 11 - DNA, 28 bp, Artificial
  probe
ctcaaaactgacttacgagtcacgtttg

SEQ ID NO: 12 - DNA, 30 bp, Artificial
  probe
ttaatatctcaaaactgacttacgagtcac

SEQ ID NO: 13 - DNA, 25 bp, Artificial
  probe
aaattgaaacacagaacaacgaaag

SEQ ID NO: 14 - DNA, 26 bp, Artificial
  probe
gagtgtacctgaaagggttcactgcg

SEQ ID NO: 15 - DNA, 30 bp, Artificial
  probe
ataaaagaaaacgtttagcagacaatgagt

SEQ ID NO: 16 - DNA, 30 bp, Artificial
  probe
cgtttcctgtaggatggaaacatagattaa

SEQ ID NO: 17 - DNA, 30 bp, Artificial
  probe
gtttgtctttaagacaaacaccaaaataac

SEQ ID NO: 18 - DNA, 30 bp, Artificial
  probe
attgatagttcacaagcgcaagcttgtagc

SEQ ID NO: 19 - DNA, 27 bp, Artificial
  probe
gctgtatgacatcgtgaatagggcatt

SEQ ID NO: 20 - DNA, 27 bp, Artificial
  probe
ttctgacttaagatgtcggaagcgttt

SEQ ID NO: 21 - DNA, 18 bp, Artificial
  primer
ttgtacacaccgcccgtc

SEQ ID NO: 22 - DNA, 20 bp, Artificial
  primer
gggttyccccrttcrgaaat

(19) United States
(12) Patent Application Publication
(10) Pub. No.: US 2006/0228718 A1
(43) Pub. Date: Oct. 12, 2006

(54) Method for rapidly identifying bacteria and kit thereof

(75) Inventors: Tsung Chain Chang, Yangmei Township (TW); Miao Chu Lin, Yujing Township (TW); Yu-Cheng Lin, Tainan City (TW); Hong-Ping Lin, Taipei City (TW)

Correspondence Address: LADAS & PARRY, 26 WEST 61ST STREET, NEW YORK, NY, 10023, US

(73) Assignee: NATIONAL CHENG KUNG UNIVERSITY

(21) Appl. No.: 11/103,643

(22) Filed: Apr. 11, 2005

(51) Int. Cl. C12Q 1/68

(52) U.S. Cl. 435/6

(57) ABSTRACT

The present invention provides a method for identifying bacteria in a sample comprising (a) obtaining the intergenic spacer region separating the 16S and 23S rDNA of bacteria in the sample; and (b) hybridizing the intergenic spacer region with at least one specific probe. A kit for identifying bacteria in a sample is also provided.

### BACKGROUND OF THE INVENTION

1. Field of the Invention

The invention relates to a method for identifying bacteria; particularly, to a method for identifying bacteria using specific probes.

2. Description of the Related Art

Conventional methods for the identification of microorganism are based on biochemical assay. For example, for the identification of food sanitation microorganisms, the method comprises selective enrichment of the interest microorganisms followed by isolation on different selective media. Suspicious colonies on selective agar plates are tested by a battery of biochemical reactions for species confirmation. For example, lauryl tryptose broth (LST) and EC broth are used for selective enrichment of Escherichia coli in food samples, followed by isolation of suspicious E. coli on Levine's eosin-methylene blue (L-EMB) agar (Hitchins, A. D. et al. 1995, pp. 4.01-4.29 In Bacteriological analytical manual, 8th ed. AOAC International, Gaithersburg, Md.). The suspicious colonies (dark centered, with or without metallic sheen) are then confirmed by tests of Gram stain, indole production, production of Voges-Poskauer (VP)- and methyl red-reactive compounds, utilization of citrate, and gas production from LST broth. The whole confirmation procedures may take as long as four days (Hitchins, A. D. et al. 1995). In addition to the lengthy procedures and high cost for performing the biochemical tests, it is difficult for a food microbiology laboratory to maintain a large set of various media for confirmation tests. It is estimated that at least 50 media are required to identify suspicious colonies of Bacillus cereus, E. coli, Listeria monocytogenes, Pseudomonas aeruginosa, Salmonella, Staphylococcus aureus, and Vibrio parahaemolyticus. (Andrews, W. H. et al. 1995, pp. 5.01-5.20. In Bacteriological analytical manual, 8th ed. AOAC International, Gaithersburg, Md.; Bennette, R. W., and G. A. Lancette. 1995. pp. 12.01-12.05. In Bacteriological analytical manual, 8th ed. AOAC International, Gaithersburg, Md.; Elliot, E. L. et al. 1995. pp. 9.01-9.27. In Bacteriological analytical manual, 8th ed. AOAC International, Gaithersburg, Md.; Hitchins, A. D. et al, 1995; pp. 4.01-4.29. In Bacteriological analytical manual, 8th ed. AOAC International, Gaithersburg, Md.; Hitchins, A. D. 1995. pp. 10.01-10.13. In Bacteriological analytical manual, 8th ed. AOAC International, Gaithersburg, Md.; Rhodehamel, E. J., and S. M. Harmon. 1995. pp. 14.01-14.08. In Bacteriological analytical manual, 8th ed. AOAC International, Gaithersburg, Md.; Covert, T. C. 1992. pp. 9-31-932. In R. E. Greenberg, L. S. Clesceri, and A. D. Eaton (ed.). Standard methods for the examination of water and wastewater, 18th ed. American Public Health Association, Washington, D.C.).

Numerous studies have been reported by using polymerase chain reaction (PCR) (Ferretti, R. et al. 2001. Appl. Environ. Microbiol. 67:977-978; Lee, C. Y. et al. 1995. Appl. Environ. Microbiol. 61:1311-1317) to identify (or detect) a variety of food microorganisms. For PCR, different primers and thermal cycling conditions are normally required to identify different species. For this reason, a series of individual PCRs should be run in parallel or sequentially to identify different microorganisms. Multiplex PCR may be effective for a limited number of organisms (Brasher, C. W. et al. 1998. Curr. Microbiol. 37:101-107; Gilbert, C. et al. 2003. Mol. Cell. Probes 17:135-138). However, as more primers are used, the sensitivity decreases and the chance that two unrelated primers form spurious products increases.

The 23S rDNA has been utilized to develop an oligonucleotide array for identification of bacteria in positive blood cultures (Anthony, R. M. et al. 2000. J. Clin. Microbiol. 38:781-788). Due to the high level of homology of this gene in different bacteria, cross-hybridization between many species was observed. By using the variable regions of 18S rDNA, Wu et al. (Wu, Z. et al. 2003. Appl. Environ. Microbiol. 69:5389-5397) developed 33 oligonucleotide probes to detect a variety of airborne fungi by slot hybridization method. However, it was found that many species hybridized to probes designed for a single species. Volokhov et al. (Volokhov, D. et al. 2003. J. Clin. Microbiol. 41:4071-4080) used a complex oligonucleotide array encompassing 72 probes designed from five genes (fur, glyA, cdtABC, ceuB-C, and fliY) to differentiate Campylobacter jejuni, C. coli, C. lari, and C. upsaliensis. A microarray was also developed for the detection and identification of Mycobacterium species based on the sequence divergence of DNA gyrase B subunit genes. The Mycobacterium microarray was hybridized with fluorescently labeled RNA to yield a pattern of positive spots (Fukushima, M. et al. 2003. J. Clin. Microbiol. 41:2605-2615). The methods mentioned above are very complicated or the sensitivity and specificity thereof are not satisfied. A rapid and convenient method is needed in this field for identifying microorganism in a sample.

### SUMMARY OF THE INVENTION

The invention provides a method for identifying bacteria in a sample comprising hybridizing an intergenic spacer region separating the 16S and 23S rDNA of the bacteria in the sample with at least one probe selected from the group consisting of BC2 (SEQ ID NO. 1), BC4 (SEQ ID NO. 2), EC5 (SEQ ID NO. 3), EC7 (SEQ ID NO. 4), LM2 (SEQ ID NO. 5), LM4 (SEQ ID NO. 6), LM6 (SEQ ID NO. 7), LM5 (SEQ ID NO. 8), PA2 (SEQ ID NO. 9), PA6 (SEQ ID NO. 10), SAL (SEQ ID NO. 11), SAL3 (SEQ ID NO. 12), SAL6 (SEQ ID NO. 13), SAL7 (SEQ ID NO. 14), SA4 (SEQ ID NO. 15), SA5 (SEQ ID NO. 16), VP4 (SEQ ID NO. 17), VP6 (SEQ ID NO. 18), the complement thereof, and.

If the intergenic spacer region is hybridized with BC2, BC4, the complement thereof, or the variant thereof, the bacterium is identified as Bacillus cereus;

if the intergenic spacer region is hybridized with EC5, EC7, the complement thereof, or the variant thereof, the bacterium is identified as Escherichia coli;

if the intergenic spacer region is hybridized with LM2, LM4, LM6, LM5, the complement thereof, or the variant thereof, the bacterium is identified as Listeria monocytogenes;

if the intergenic spacer region is hybridized with PA2, PA6, the complement thereof, or the variant thereof, the bacterium is identified as Pseudomonas aeruginosa;

if the intergenic spacer region is hybridized with two or more probes of SAL, SAL3, SAL6, SAL7, the complement thereof, or the variant thereof, the bacterium is identified as Salmonella spp.;

if the intergenic spacer region is hybridized with SA4, SA5, the complement thereof, or the variant thereof, the bacterium is identified as Staphylococcus aureus; and

if the intergenic spacer region is hybridized with VP4, VP6, the complement thereof, or the variant thereof, the bacterium is identified as Vibrio parahaemolyticus.

The invention also provides a kit for identifying bacteria in a sample comprising at least one probe selected from the group consisting of BC2, BC4, EC5, EC7, LM2, LM4, LM6, LM5, PA2, PA6, SAL, SAL3, SAL6, SAL7, SA4, SA5, VP4, and VP6, the complement thereof, and the variant thereof.

### BRIEF DESCRIPTION OF THE DRAWINGS

FIG. 1. Amplification of bacterial ITS regions and separation of the PCR products by 2% agarose gel electrophoresis. Lanes M: 100-bp ladder; 1: Xanthobacter flavus BCRC 12271; 2: Salmonella ser. Paratyphi A BCRC 12949; 3: Salmonella ser. Typhimurium BCRC 11490; 4: Escherichia coli BCRC 15486; 5: Listeria monocytogenes BCRC 14846; 6: Bacillus cereus BCRC 10250; 7: Staphylococcus aureus BCRC 15299; 8: Pseudomonas aeruginosa ATCC 27853; and 9: Vibrio parahaemolyticus BCRC 10806.

FIG. 2. Hybridization of target and non-target microorganisms to the oligonucleotide arrays. The positions of oligonucleotide probes are indicated in the upper right-hand array and their sequences are indicated in Table 2. The strain(s) tested is indicated at the bottom of each array. PM: position marker (primer 6R with its 5′ end labeled with digoxigenin). PC1 and PC2: positive control probes designed from the ITS region of X. flavus. NC: negative control (tracking dye only).

### DETAILED DESCRIPTION OF THE INVENTION

The present invention provides a method for identifying bacteria in a sample, which comprises hybridizing an intergenic spacer region separating the 16S and 23S rDNA of the bacteria in the sample with at least one probe selected from the group consisting of BC2 (SEQ ID NO. 1), BC4 (SEQ ID NO. 2), EC5 (SEQ ID NO. 3), EC7 (SEQ ID NO. 4), LM2 (SEQ ID NO. 5), LM4 (SEQ ID NO. 6), LM6 (SEQ ID NO. 7), LM5 (SEQ ID NO. 8), PA2 (SEQ ID NO. 9), PA6 (SEQ ID NO. 10), SAL (SEQ ID NO. 11), SAL3 (SEQ ID NO. 12), SAL6 (SEQ ID NO. 13), SAL7 (SEQ ID NO. 14), SA4 (SEQ ID NO. 15), SA5 (SEQ ID NO. 16), VP4 (SEQ ID NO. 17), VP6 (SEQ ID NO. 18), the complement thereof, and the variant thereof.

If the intergenic spacer region is hybridized with BC2, BC4, the complement thereof, or the variant thereof, the bacterium is identified as Bacillus cereus;

if the intergenic spacer region is hybridized with EC5, EC7, the complement thereof, or the variant thereof, the bacterium is identified as Escherichia coli;

if the intergenic spacer region is hybridized with LM2, LM4, LM6, LM5, the complement thereof, or the variant thereof, the bacterium is identified as Listeria monocytogenes;

if the intergenic spacer region is hybridized with PA2, PA6, the complement thereof, or the variant thereof, the bacterium is identified as Pseudomonas aeruginosa;

if the intergenic spacer region is hybridized with two or more probes of SAL, SAL3, SAL6, SAL7, the complement thereof, or the variant thereof, the bacterium is identified as Salmonella spp.;

if the intergenic spacer region is hybridized with SA4, SA5, the complement thereof, or the variant thereof, the bacterium is identified as Staphylococcus aureus; and

if the intergenic spacer region is hybridized with VP4, VP6, the complement thereof, or the variant thereof, the bacterium is identified as Vibrio parahaemolyticus.

The intergenic spacer (ITS) region separating the 16S and 23S rDNA has been suggested as a good candidate for bacterial species identification (Chen, C. C. et al. 2004. J. Clin. Microbiol. 42:2651-2657; Roth, A. et al. 1998. J. Clin. Microbiol. 36:139-147) and strain typing. Sequences of the ITS regions have been found to have low intraspecies variation and high interspecies divergence (Chen, C. C. et al. 2004; Roth, A. et al. 1998; Whiley, R. A. et al. 1995. Microbiology 141:1461-1467). The present invention provides a panel of oligonucleotide probes to identify six food sanitation microorganisms (B. cereus, E. coli, L. monocytogenes, Salmonella spp., S. aureus, and V. parahaemolyticus) and one indicator microorganism of natural waters and swimming pool waters (P. aeruginosa). Therefore, in one preferred embodiment of the invention, the sample is food or water.

Prior to the hybridization, the intergenic spacer region separating the 16S and 23S rDNA of bacteria in the sample can be amplified in a polymerase chain reaction to facilitate convenient manipulation. Preferably, the intergenic spacer region separating the 16S and 23S rDNA of bacteria in the sample is amplified with bacteria-specific universal primers in order to avoid forming spurious products due to using multiple primers and in order to increase sensitivity. The sequence of the universal primer is well disclosed. In one embodiment of the invention, the bacteria-specific universal primers comprise 2F primer (SEQ ID NO. 21) as designed by Relman (Relman, D. A. 1993. pp. 489-495. In D. H. Persing, T. F. Smith, F. C. Tenover, and T. J. White (ed.), Diagnostic molecular microbiology, American Society for Microbiology, Washington, D.C.) and 6R primer (SEQ ID NO. 22) as designed by Gurtler and Stanisich (Gurtler, V., and V. A. Stanisich. 1996.).

According to the invention, BC2 and BC4 are B. cereus-specific probes. The sensitivity and specificity for the identification of B. cereus as shown in the example of the invention are 100% (26/26) and 98.9% (523/529), respectively. There are two species (B. mycoides and B. thuringiensis) of the B. cereus group found to cross-hybridize to the probes BC2 and BC4. However, combining the present invention with conventional methods is successful for identifying B. cereus. For example, old cultures (48-72 h) of B. mycoides is differentiated from B. cereus by production of colonies with long hair or root-like structures (rhizoid growth) (Rhodehamel, E. J., and S. M. Harmon. 1995.). In addition, old cultures (48-72 h) of B. thuringiensis usually produce protein toxin crystals that can be detected by staining with basic fushsin. B. cereus does not produce protein toxin crystals.

According to the invention, EC5 and EC7 are E. coli-specific probes. Both the sensitivity (48/48) and specificity (507/507) for the identification of E. coli as shown in the example of the invention are 100%. Some species in Enterobacteriaceae (Escherichai fergusonii, Shigella boydii, S. flexneri, and S. sonnei) are found to cross-hybridize to probes EC5 and EC7. However, combining the present invention with conventional methods is successful for identifying E. coli. For example, no strains (100%) of E. fergusonii can ferment lactose, and only 1 to 2% of Shigella is lactose fermenters (Farmer, J. J. III. 2003. pp. 636-653. In P. R. Murray, E. J. Baron, J. H. Jorgensen, M. A. Pfaller, and R. H. Yolken (ed.), Manual of clinical microbiology, 8th ed. American Society for Microbiology, Washington, D.C.). Such cross-reacting species are unable to produce suspicious colonies on L-EMB agar used for isolation of E. coli from food (Hitchins, A. D. et al. 1995.).

According to the invention, LM2, LM4, LM5 and LM6 are L. monocytogenes-specific probes. The sensitivity and specificity for the identification of L. monocytogenes as shown in the example of the invention are 100% (40/40) and 99.6% (513/515), respectively. It is found that the ITS sequence of L. monocytogenes (GenBank accession no. U44063) has similarities of 0.90, 0.92, 0.89, and 0.86, respectively, with L. welshimeri (U78982), L. innocua (U57914), L. ivanovii (U78981), and L. seeligeri (U78984). Except L. grayi and L. murrayi, other Listeria tested in the example of the invention cross-hybridized to at least two of the four probes. L. innocua is the only species cross-hybridized to all the four probes, and cannot be differentiated from L. monocytogenes by the hybridization assay. An additional beta-hemolysis test can be used to differentiate L. monocytogenes from L. innocua. The former is hemolytic on sheep blood agar, whereas the latter is nonhemolytic (Bille, J. et al. 2003. pp. 461-471. In P. R. Murray, E. J. Baron, J. H. Jorgensen, M. A. Pfaller, and R. H. Yolken (ed.), Manual of clinical microbiology, 8th ed. American Society for Microbiology, Washington, D.C.; Hitchins, A. D. 1995.).

According to the invention, PA2 and PA6 are P. aeruginosa-specific probes. Both the sensitivity (29/29) and specificity (526/526) for the identification of P. aeruginosa as shown in the example of the invention are 100%.

According to the invention, SAL, SAL3, SAL6 and SAL7 are Salmonella spp.-specific probes. Both the sensitivity (51/51) and specificity (504/504) for the identification of Salmonella spp. as shown in the example of the invention are 100%. S. ser. Paratyphi A BCRC 12949 is found to have three ITS fragments whereas S. ser. Typhimurium BCRC 11490 has only one fragment. The hybridization results demonstrate that most strains of Salmonella hybridized to all four probes, whereas some strains only hybridized to two or three of the four probes. For this reason, if a strain hybridized to at least two of the four probes, the microorganism is recognized as a strain of Salmonella.

According to the invention, SA4 and SA5 are S. aureus-specific probes. The sensitivity and specificity for the identification of S. aureus as shown in the example of the invention are 95% (38/40) and 100% (515/515), respectively. Two of the 40 strains of S. aureus tested in the example are unable to hybridize to both SA4 and SA5 probes. The identity of the two strains as S. aureus can be further reconfirmed by conventional methods (Bennette, R. W., and G. A. Lancette. 1995.).

According to the invention, VP4 and VP6 are V. parahaemolyticus-specific probes. The sensitivity and specificity for the identification of V. parahaemolyticus as shown in the example of the invention are 100% (43/43) and 99.4% (509/512), respectively. Strains of V. alginolyticus, V. harveyi, and V. mimicus are also found to hybridize to probes VP4 and VP6. However, most strains (99%) of V. alginolyticus can utilize sucrose and produce yellow colonies on TCBS agar (Farmer, J. J. III et al. 2003. pp. 706-718. In P. R. Murray, E. J. Baron, J. H. Jorgensen, M. A. Pfaller, and R. H. Yolken (ed.), Manual of clinical microbiology, 8th ed. American Society for Microbiology, Washington, D.C.) that is not typical of V. parahaemolyticus (green colonies on TCBS) (Elliot, E. L. et al. 1995.). Two strains of V. harveyi and one strain of V. mimicus produced false-positive reactions in the example of the invention. About 50% strains of V. harveyi and 100% strains of V. mimicus are sucrose non-fermenters (Farmer, J. J. III et al. 2003) and these strains may produce false-positive hybridization reactions. A simple test is suggested to be used to differential V. mimicus from V. parahaemolyticus. The former grows in nutrient broth without NaCl whereas NaCl must be added to the broth to support the growth of V. parahaemolyticus (Farmer, J. J. III et al. 2003). In addition, 90% of V. parahaemolyticus strains can produce lipase but all strains of V. harveyi are lipase-negative (Farmer, J. J. III et al 2003). The lipase test can be used to differentiate V. parahaemolyticus from V. harveyi.

According to the invention, at least one probe selected from the group consisting of BC2, BC4, EC5, EC7, LM2, LM4, LM5, LM6, PA2, PA6, SAL, SAL3, SAL6, SAL7, SA4, SA5, VP4, VP6, the complement thereof, and the variant thereof is used. The combination of primers can be determined by artisans skilled in this field for identifying different microorganisms. Preferably, the probes used comprise BC2, BC4, EC5, EC7, LM2, LM4, LM5, LM6, PA2, PA6, SAL, SAL3, SAL6, SAL7, SA4, SA5, VP4, VP6, the complement thereof, and the variant thereof, and the seven microorganisms can be identified in a single detection.

As used herein, the term “variant” refers to a probe having any part of the sequences detailed above, having a length of at least about 15 nucleotide bases, and being capable of hybridizing to the nucleic acid of intergenic spacer region separating the 16S and 23S rDNA of the bacteria. For example, a sequence of nucleic acid which begins in the middle of one of above-described sequences and extends further than the end of that sequence, and which detects the intergenic spacer region separating the 16S and 23S rDNA of the bacteria is suitable for use in the invention.

Preferably, the probe(s) for hybridization is (are) coated on a substrate. The material of substrate and the method of coating are both well established in this field. For example, a membrane or a glass slide is considered as a good substrate according to the invention. More preferably, the hybridization is carried out by using a microarray; wherein the probes are microarrayed on a solid or a chip in order to identify multiple microorganisms in one manipulation. Any detection methods for gene expression commonly used in the art can be used in the invention.

For easy detection, the intergenic spacer region separating the 16S and 23S rDNA of the bacteria in the sample is preferably labeled. The method for labeling the region is well known to the artisans skilled in this field. For example, when the polymerase chain reaction is performed for amplifying the region, a labeled primer (such as labeled with digoxigenin) or labeled dNTP is used to incorporate a maker into the product.

In a preferred embodiment of the invention, the method further comprises a positive control step. Any established probe for identification a known microorganism is suitable for the application in the positive control step. In a more preferred embodiment of the invention, the positive control step is hybridizing an intergenic spacer region separating the 16S and 23 S rDNA of Xanthobacter flavus with at least one probe of PC 1 (SEQ ID NO. 19) and PC2 (SEQ ID NO. 20).

The important feature of the present method is that, instead of using a different battery of biochemical reactions to identify each microorganism, it uses a single protocol for the seven target species. An additional prominent feature of the technique is that multiple different suspicious species can be simultaneously identified on a single array and this may save much time, expense, space, and manpower.

The good performance of the method according to the invention might be due to the facts that the ITS sequence is species-specific and a few cross-reacting bacteria may not produce typical colonies on the selective media as the target microorganisms. It is also important that multiple probes are designed for each organism and the chance of a non-target bacterium to hybridize to all probes designed for the organism is quite low. The present method can be continually extended by adding further oligonucleotides to the panel without significantly increasing the cost or complexity.

The present invention also provides a kit for identifying bacteria in a sample comprising at least one probe selected from the group consisting of BC2, BC4, EC5, EC7, LM2, LM4, LM5, LM6, PA2, PA6, SAL, SAL3, SAL6, SAL7, SA4, SA5, VP4, VP6, the complement thereof, and the variant thereof.

According to the invention, the kit further comprises reagents for obtaining the intergenic spacer region separating the 16S and 23S rDNA of bacteria. The components of the reagents in addition to the probes of the kit are well known to the artisans skilled in this field. In a preferred embodiment of the invention, the reagents are for polymerase chain reaction. In a more preferred embodiment of the invention, the kit further comprises bacteria-specific universal primers. In another preferred embodiment of the invention, the kit further comprises reagents for hybridization.

The following examples are given for the purpose of illustration only and are not intended to limit the scope of the present invention.

### EXAMPLE

Bacterial strains. A total of 555 strains including seven target bacteria (277 strains) and non-target bacteria (278 strains) were used in this example. The target microorganisms included 26 strains of B. cereus; 48 strains of E. coli, 40 strains of L. monocytogenes, 29 strains of P. aeruginosa, 51 strains of Salmonella, 40 strains of S. aureus, and 43 strains of V. parahaemolyticus as shown in Table 1. Reference strains were obtained from the American Type Culture Collection (ATCC, Manassa, Va.) and the Bioresources Collection and Research Center (BCRC, Hsinchu, Taiwan). Non-reference strains were isolated from a variety of sources including foods, environmental, and clinical specimens. All bacteria except Vibrio spp. were grown at 37° C. on tryptic soy agar (TSA) for 20 to 24 h before DNA extraction. Strains of Vibrio spp. were grown at 37° C. on TSA-1% NaCl.

TABLE 1
Bacterial strains used in this invention

| | No. of | | |
|---|---|---|---|
| | reference | No. of non- | |
| Species | strain^{a} | reference strain^{b} | Total |
| Bacillus cereus | 24 | 2 | 26 |
| Escherichia coli | 24 | 24 | 48 |
| Listeria monocytogenes | 32 | 8 | 40 |
| Pseudomonas aeruginosa | 2 | 27 | 29 |
| Salmonella spp.^{c} | 42 | 9 | 51 |
| Staphylococcus aureus | 26 | 14 | 40 |
| Vibrio parahaemolyticus | 5 | 38 | 43 |
| Abiotrophia defectiva | 1 | | 1 |
| Acinetobacter baumannii | 1 | 10 | 11 |
| Acinetobacter calcoaceticus | 5 | | 5 |
| Acinetobacter haemolyticus | 1 | | 1 |
| Acinetobacter lwoffii | 1 | | 1 |
| Alcaligenes faecalis | 1 | | 1 |
| Alcaligenes xylosoxidans | 2 | | 2 |
| Aeromonas hydrophila | 1 | | 1 |
| Aeromonas sobria | | 1 | 1 |
| Bacillus amyloliquefaciens | 1 | | 1 |
| Bacillus circulans | 2 | | 2 |
| Bacillus coagulans | 2 | | 2 |
| Bacillus firmus | 2 | | 2 |
| Bacillus licheniformis | 2 | | 2 |
| Bacillus mycoides | 2 | | 2 |
| Bacillus megaterium | 1 | | 1 |
| Bacillus polymyxa | 1 | | 1 |
| Bacillus psychrophilus | 1 | | 1 |
| Bacillus pumilus | 2 | | 2 |
| Bacillus sphaericus | 3 | | 3 |
| Bacillus stearothermophilus | 1 | | 1 |
| Bacillus subtilis | 2 | | 2 |
| Bacillus thuringiensis | 4 | | 4 |
| Burkholderia cepacia | 1 | | 1 |
| Citrobacter freundii | | 5 | 5 |
| Citrobacter diversus | 1 | | 1 |
| Edwardsiella tarda | 2 | | 2 |
| Enterobacter aerogenes | 1 | | 1 |
| Enterobacter agglomerans | | 2 | 2 |
| Enterobacter cloacae | | 4 | 4 |
| Escherichia blattae | 3 | | 3 |
| Escherichia fergusonii | 5 | | 5 |
| Escherichia hermannii | 1 | | 1 |
| Escherichia vulneris | 3 | | 3 |
| Enterococcus sp. | 2 | | 2 |
| Granulicatella adiacens | 1 | | 1 |
| Klebsiella oxytoca | | 1 | 1 |
| Klebsiella pneumoniae | 1 | 3 | 4 |
| Listeria grayi | 3 | | 3 |
| Listeria innocua | 4 | | 4 |
| Listeria ivanovii | 2 | | 2 |
| Listeria murrayi | 2 | | 2 |
| Listeria seeligeri | 2 | | 2 |
| Listeria welshimeri | 7 | | 7 |
| Micrococcus sp. | | 2 | 2 |
| Plesiomonas shigelloides | 1 | | 1 |
| Proteus mirabilis | 1 | | 1 |
| Proteus vulgaris | | 1 | 1 |
| Pseudomonas alcaligenes | 3 | | 3 |
| Pseudomonas fluorescens | 3 | | 3 |
| Pseudomonas mendocina | 2 | | 2 |
| Pseudomonas | 2 | | 2 |
| pseudoalcaligenes | | | |
| Pseudomonas putida | 2 | | 2 |
| Pseudomonas stutzeri | 1 | | 1 |
| Serratia marcescens | 1 | 2 | 3 |
| Shigella boydii | 2 | | 2 |
| Shigella flexneri | 2 | 2 | 4 |
| Shigella sonnei | 1 | 2 | 3 |
| Stenotrophomonas maltophilia | | 9 | 9 |
| Staphylococcus auricularis | 2 | | 2 |
| Staphylococcus capitis | 4 | 1 | 5 |
| Staphylococcus caseolyticus | 2 | | 2 |
| Staphylococcus cohnii | 2 | | 2 |
| Staphylococcus delphini | 1 | | 1 |
| Staphylococcus epidermidis | 3 | 1 | 4 |
| Staphylococcus haemolyticus | 3 | 2 | 5 |
| Staphylococcus hominis | 1 | | 1 |
| Staphylococcus intermedius | 2 | 1 | 3 |
| Staphylococcus lentus | 1 | | 1 |
| Staphylococcus saprophyticus | 2 | 1 | 3 |
| Staphylococcus sciuri | 3 | | 3 |
| Staphylococcus simulans | | 1 | 1 |
| Staphylococcus xylosus | 5 | | 5 |
| Staphylococcus warneri | 3 | | 3 |
| Streptococcus agalactiae | 1 | | 1 |
| Streptococcus anginosus | 1 | | 1 |
| Streptococcus bovis | 1 | 2 | 3 |
| Streptococcus constellatus | 1 | | 1 |
| Streptococcus equinus | 1 | | 1 |
| Streptococcus equisimilis | 1 | | 1 |
| Streptococcus mitis | 1 | | 1 |
| Streptococcus mutans | 1 | | 1 |
| Streptococcus oralis | 1 | | 1 |
| Streptococcus pneumoniae | 1 | 2 | 3 |
| Streptococcus pyogenes | 1 | | 1 |
| Streptococcus salivarius | 1 | | 1 |
| Streptococcus sanguis | 1 | | 1 |
| Streptococcus uberis | 1 | | 1 |
| Vibrio alginolyticus | 1 | 2 | 3 |
| Vibrio campbellii | | 1 | 1 |
| Vibrio carchariae | 1 | | 1 |
| Vibrio cholerae non-O1 | | 12 | 12 |
| Vibrio damsela | 1 | | 1 |
| Vibrio fluvialis | | 1 | 1 |
| Vibrio furnissii | 1 | 2 | 3 |
| Vibrio hollisae | 1 | 1 | 2 |
| Vibrio harveyi | 1 | 3 | 4 |
| Vibrio metschnikovii | 1 | | 1 |
| Vibrio mediterranei | | 1 | 1 |
| Vibrio mimicus | 1 | 2 | 3 |
| Vibrio nigrapulchritudo | | 1 | 1 |
| Vibrio orientalis | | 1 | 1 |
| Vibrio proteolyticus | 1 | 1 | 2 |
| Vibrio splendidus | | 1 | 1 |
| Vibrio tubiashii | | 1 | 1 |
| Vibrio vulnificus | 2 | 29 | 31 |
| Yersinia enterocolitica | 1 | | 1 |
| Total | | | 555 |

^{a}Reference strains were obtained from either the American Type Culture Collection (ATCC, Manassa, VA) or the Bioresources Collection and Research Center (BCRC, Hsinchu, Taiwan).
^{b}Non-reference strains were isolated from foods, environmental and clinical specimens.
^{c}Thirty five serotypes were included.

DNA preparation. The boiling method described by Vaneechoutte et al. (Vaneechoutte, M. et al. 1995. J. Clin. Microbiol. 33:11-15) was followed to extract DNA from bacteria. Briefly, one colony of pure cultures was suspended in 50 μL of sterilized water and heated at 100° C. for 15 min in a heating block. After centrifugation in a microfuge (5,000×g for 3 min), the supernatant containing bacterial DNA was stored at −20° C. for further use.

Amplification of the ITS regions and nucleotide sequence determination. The ITS regions of bacteria were amplified by PCR. The bacteria-specific universal primers 2F (5′-TTG TAC ACA CCG CCC GTC-3′, SEQ ID No. 21) and 6R (5′-GGG TTY CCC CRT TCR GAA AT-3′, Y is C or T, and R is A or G, SEQ ID No. 22) were used to amplify a DNA fragments containing the ITS region. The 5′-end of primer 2F is located at position 1390 of the 16S rDNA and the 5′-end of primer 6R at position 111 downstream of the 5′-end of the 23S rDNA (E. coli numbering). PCR was performed with 5 μL (1 to 5 ng) of template DNA in a total reaction volume of 50 μL consisting of 10 mM Tris-HCl (pH 8.3), 50 mM KCl, 1.5 mM MgCl₂, 0.8 mM deoxyribonucleoside triphosphates (0.2 mM each), 1 μM primer 2F, 2 μM primer 6R, 1 U Taq DNA polymerase, and 50 μL of a mineral oil overlay. PCR program consisted of eight cycles of denaturation (94° C., 2 min), annealing (55° C., 1 min), and extension (72° C., 1 min), followed by 30 cycles of denaturation (94° C., 1 min), annealing (60° C., 1 min), and extension (72° C., 1 min), and a final extension step at 72° C. for 3 min. To prepare digoxigenin-labeled PCR products, the reverse primer 6R was labeled with digoxigenin at its 5′ end (MDBio® Inc., Taipei, Taiwan) and digoxigenin-11-dUTP (Roche®, Mannheim, Germany) was added to the PCR reaction mixture at a final concentration of 10 μM. DNA extracted from X. flavus BCRC 12271 was used as a positive control. An OmniGen thermal cycler (Hybaid® Limited, Middlesex, UK) was used for PCR.

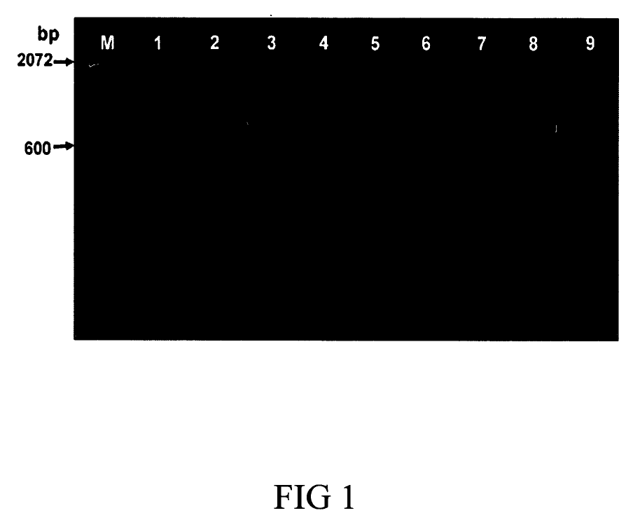

For strains (X. flavus BCRC 12271, Salmonella ser. Typhimurium BCRC 11490, and P. aeruginosa ATCC 27853) that produced a single PCR product as shown in FIG. 1, the amplicons were directly sequenced in both directions on a model 377 sequencing system (Applied Biosystems®, Taipei, Taiwan) with the BigDye™ Terminator Cycle Sequencing kit (version 3.1, Applied Biosystems). For strains (E. coli BCRC 15486, L. monocytogenes BCRC 14486, Salmonella ser. Paratyphi A BCRC 12949, and V. parahaemolyticus BCRC 10806) that produced multiple PCR products, the shortest fragments were removed from the stained agarose gels under UV light. DNA in the gels (approximately 300 mg for each strain) was extracted with a Gel-M™ gel extraction kit (Viogene®, Taipei, Taiwan) and sequenced. The size of PCR products ranged from approximately 400 bp (B. cereus) to 950 bp (X. flavus).

The ITS regions of ten strains of each of the seven target microorganisms were also amplified by PCR from colonies grown on selective media. Selective media used were as follows: mannitol-egg yolk-polymyxin (MYP) agar for B. cereus (MYP) (Rhodehamel, E. J., and S. M. Harmon. 1995.), Levine's eosin-methylene blue (L-EMB) agar for E. coli (Hitchins, A. D. et al. 1995.), lithium chloride-phenylethanol-moxalactam (LPM) agar and Oxford medium (OXA) for L. monocytogenes (Hitchins, A. D. 1995.), bismuth sulfite (BS) agar, xylose lysine desoxycholate (XLD) agar, and Hektoen enteric (HE) agar for Salmonella spp. (Andrews, W. H. et al. 1995.), Baird-Parker agar (BPA) for S. aureus (Bennette, R. W., and G. A. Lancette. 1995.), thiosulfate-citrate-bile salts-sucrose (TCBS) for V. parahaemolyticus (Elliot, E. L. et al. 1995.), and modified M-PA agar for P. aeruginosa (Covert, T. C. 1992.).

Probe design. Species-specific oligonucleotide probes for the seven food microorganisms were designed from sequences of the ITS regions. Alignments were performed among the ITS sequences obtained in this study and those available from GenBank. To create an alignment of multiple ITS sequences, the PrettyBox command of the Wisconsin Genetics Computer Group (GCG) Package (Version 10.3, Accelrys Inc., San Diego, Calif.) was used. After screening against GenBank for homology with other bacteria and conducting preliminary hybridization tests, 20 probes including two positive controls (probes designed from X. flavus) were synthesized (MDBio®, Taipei, Taiwan) for further evaluation. The probes are shown in Table 2.

TABLE 2
Oligonucleotide probes used in this invention

| | | | | |
|---|---|---|---|---|
| | | | SEQ | GenBank |
| Micro- | Probe | | ID | accession |
| organism | code^{a} | Probe sequence (5′-3′) | No. | no. |

| | | | | |
|---|---|---|---|---|
| B. cereus | BC2 | AAAGTTTCCGTGTTTCGTT | 1 | AF267900^{b} |
| NCTC9620 | | TTGTTCAG | | |
| | BC4 | CTGTTCATCAATATAAGTT | 2 | AF267900^{b} |
| | | TCCGTGTTTCG | | |
| E. coli | EC5 | ACGGCAAATTTGAAGAGG | 3 | AY684796 |
| BCRC 15486 | | TTTTAACTACAT | | |
| | EC7 | CTGTAGTGATTAAATAAAA | 4 | AY684796 |
| | | AATACTTCAGA | | |
| L. monocyto- | LM2 | CTTCTCAGTATGTTTGTTCT | 5 | U44063^{b} |
| genes ATCC | | TCTCAGTATG | | |
| 49594 | LM4 | ACTTCTCAGTATGTTTGTTC | 6 | U44063^{b} |
| | | TTCTC | | |
| | LM6 | CATAGATAATTTATTATTT | 7 | U44063^{b} |
| | | ATGACACAAGT | | |
| L. monocyto- | LM5 | TGGATGTATCATCGCTGAT | 8 | AY684791 |
| genes BCRC | | ACGGAAAATCA | | |
| 14846 | | | | |
| P. aeruginosa | PA2 | ATTGTTGGTGTGCTGCGTG | 9 | AY684792 |
| ATCC 27853 | | ATCC | | |
| | PA6 | GAAGTAAGACTGAATGAT | 10 | AY684792 |
| | | CTCTTTCACTGG | | |
| Salmonella | A SAL | CTCAAAACTGACTTACGAG | 11 | AY684793 |
| ser. Paratyphi | | TCACGTTTG | | |
| BCRC 12949 | | | | |
| | SAL3 | TTAATATCTCAAAACTGAC | 12 | AY684793 |
| | | TTACGAGTCAC | | |
| Salmonella ser. | SAL6 | AAATTGAAACACAGAACA | 13 | AY684794 |
| Typhimurium | | ACGAAAG | | |
| BCRC 11490 | | | | |
| | SAL7 | GAGTGTACCTGAAAGGGTT | 14 | AY684794 |
| | | CACTGCG | | |
| S. aureus D46 | SA4 | ATAAAAGAAAACGTTTAG | 15 | U11773^{b} |
| | | CAGACAATGAGT | | |
| S. aureus H11 | SA5 | CGTTTCCTGTAGGATGGAA | 16 | U11784^{b} |
| | | ACATAGATTAA | | |
| V. parahaemo- | VP4 | GTTTGTCTTTAAGACAAAC | 17 | AY684795 |
| lyticus BCRC | | ACCAAAATAAC | | |
| 10806 | VP6 | ATTGATAGTTCACAAGCGC | 18 | AY684795 |
| | | AAGCTTGTAGC | | |
| Xanthobacter | PC1^{c} | GCTGTATGACATCGTGAAT | 19 | AY684797 |
| flavus BCRC | | AGGGCATT | | |
| 12271 | PC2^{c} | TTCTGACTTAAGATGTCGG | 20 | AY684797 |
| | | AAGCGTTT | | |

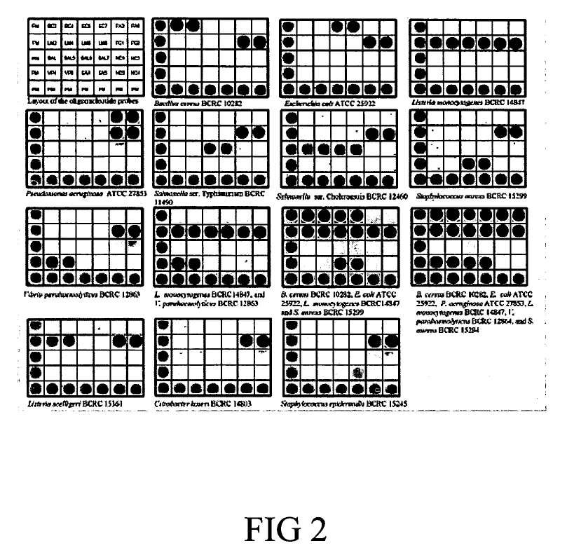

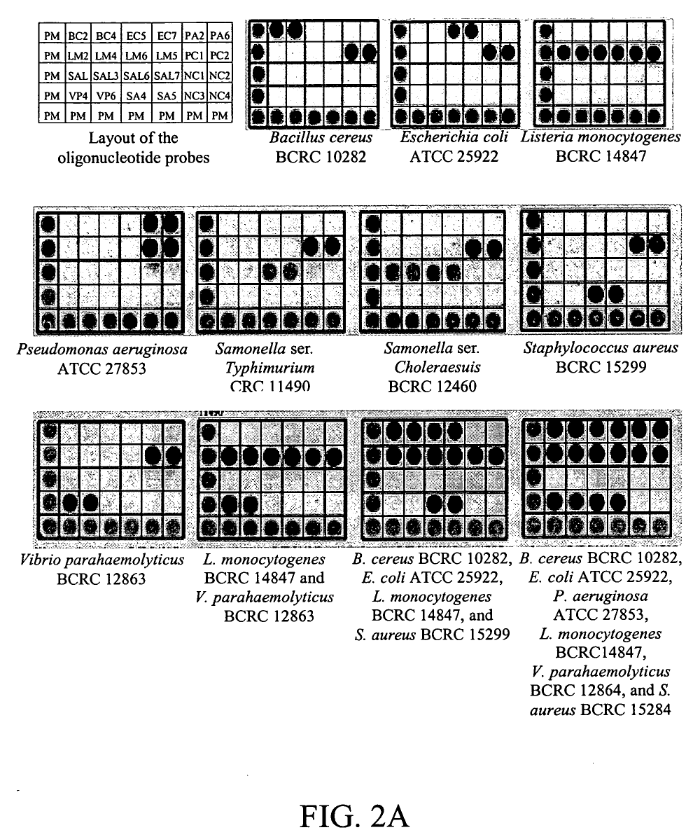

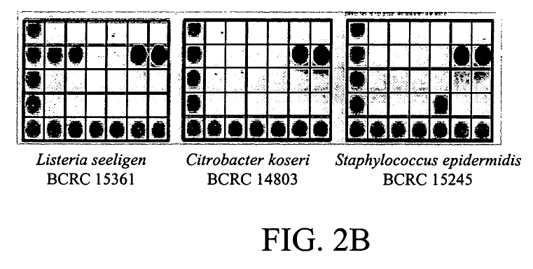

^{a}oligonucleotide probes are arranged on the array as indicated in FIG. 2.
^{b}Determined in this example.
^{c}Probes PC1 and PC2 were used as positive controls.

Production of oligonucleotide arrays. The arrays (5×8 mm) were made in batches of 20. The probes were diluted 1:1 (final concentration 5 μM) with a tracking dye solution [30% (v/v) glycerol, 40% (v/v) dimethyl sulfoxide, 1 mM ethylenediaminetetraacetic acid (EDTA, disodium salt), 0.15% (w/v) bromophenol blue, and 10 mM Tris-HCl, pH7.5]. The probe solutions were drawn into wells of a round bottom microtiter plate and spotted onto positively charged nylon membrane (Roche®) with an automatic arrayer (Wittech®, Taipei, Taiwan) using a solid pin (500 μm in diameter). The array contained 40 dots, including 18 dots for the target bacteria, two for positive (probes designed from ITS region of X. flavus) and four for negative controls (dye only). In addition, 11 dots were spotted onto the membrane at the left hand column and the bottom line with primer 6R (0.63 μM) that was labeled with digoxigenin at its 5′ end (MDBio®); the 11 dots served as a guide to localize the hybridized probes (FIG. 2). Once all the probes had been applied, the membrane was dried and exposed to shortwave UV (Stratalinker 1800; Stratagen®, La Jolla, Calif.) for 30 s. Unbound oligonucleotides were removed by two washes (2 min each) at room temperature in 0.5×SSC (1×SSC is 0.15 M NaCl plus 0.015 M sodium citrate)-0.1% sodium dodecyl sulfate (SDS). The arrays were air-dried and stored at room temperature for further use.

Hybridization protocol. Except otherwise indicated, the hybridization procedures were carried out at room temperature in a hybridization oven with a shaking speed of 60 rpm. Most reagents, except washing buffers, used for hybridization were included in the DIG Nucleic Acid Detection kit (Roche®, Cat. No. 1175041). Each array was hybridized in an individual well of a 24-well cell culture plate. The array was prehybridized at 50° C. for 2 h with 1 ml of hybridization solution [5×SSC, 1% (w/v) blocking reagent, 0.1% N-laurylsarcosine and 0.02% SDS]. The digoxigenin-labeled PCR products were heated in a boiling water bath for 5 min and immediately cooled by an ice bath. Ten microliters of denatured PCR product of the test organism and 10 μL of the denatured PCR product of positive control (amplified from X. flavus) were diluted with 0.5 ml of hybridization solution and added to each well. Hybridization was conducted at 50° C. for 90 min. After removing the non-hybridized PCR products, the array was washed four times (5 min each) in 1 ml of maleic acid buffer (0.1 M maleic acid, 0.15 M NaCl, pH 7.5), followed by blocking for 1 h with 1 ml of blocking solution [1% (w/v) blocking reagent dissolved in maleic acid buffer]. The blocking solution was then removed and 0.5 ml of alkaline phosphatase-conjugated sheep anti-digoxigenin antibodies (diluted 1:2,500 in blocking solution) was added to each well and antigen-antibody reaction was performed for 1 h. After removing the unbound antibodies, the array was washed three times (each 15 min) in 1 ml of washing solution [0.3% (v/v) Tween 20 in maleic acid buffer], followed by washing in 1 ml of detection buffer (0.1 M Tris-HCl, 0.15 M NaCl, pH 9.5) for 5 min. Finally, 0.5 ml of alkaline phosphatase substrate (stock solution of nitroblue tetrazolium chloride/5-bromo-4-chloro-3-indolylphosphate diluted 1:50 in detection buffer) was added to each well and incubated at 37° C. (without shaking). Color development was clearly visible between 15 min and 1 h after the start of the reaction.

Definition of positive reaction, sensitivity, and specificity. A strain was identified as one of the seven target microorganisms when all probes designed for the species and the positive control probes (PC1 and PC2) were hybridized except Salmonella. For Salmonella, if two of the four probes (SAL, SAL3, SAL6, and SAL7) were hybridized, the test microorganism was regarded as a strain of Salmonella. Sensitivity was defined as the number of positive hybridization reactions of a species (true positives) divided by total strains of the bacterium tested. Specificity was defined as the number of negative reactions of non-target microorganisms (true negatives) divided by total strains of these species tested. If a strain hybridized to probes designed of a species but displayed a typical colonies on selective isolation agar for the target microorganism, the strain was not considered to belong to the target species.

Nucleotide sequence accession numbers. The ITS sequences of E. coli BCRC 15486, L. monocytogenes BCRC 14846, P. aeruginosa ATCC 27853, Salmonella ser. Paratyphi A BCRC 12949, Salmonella ser. Typhimurium BCRC 11490, V. parahaemolyticus BCRC 10806, and X. flavus BCRC 12271 were submitted to GenBank. The accession numbers are listed in Table 2.

TABLE 3
Summary of hybridization results of different bacteria to
probes immobilized on the oligonucleotide array

| | | | | | | Colony |
|---|---|---|---|---|---|---|
| | No. of | | | | | morphology |
| | strains | Probe code and | | | | on selective |
| Species | tested | hybridization result^{a} | | | | agar^{b} |
| | | BC2 | BC4 | | | |
| B. cereus | 26 | + | + | | | |
| B. mycoides | 2 | + | + | | | T^{c} |
| B. thuringiensis | 4 | + | + | | | T |
| Other bacteria^{d} | 523 | − | − | | | |
| | | EC5 | EC7 | | | |
| E. coli | 48 | + | + | | | |
| E. fergusonii | 5 | + | + | | | A^{e} |
| Shigella boydii | 2 | + | + | | | A |
| S. flexneri | 4 | + | + | | | A |
| S. sonnei | 3 | + | + | | | A |
| Other bacteria^{d} | 493 | − | − | | | |
| | | PA2 | PA6 | | | |
| P. aeruginos^{a} | 29 | + | + | | | |
| Other bacteria^{d} | 526 | − | − | | | |
| | | SA4 | SA5 | | | |
| S. aureus | 38 | + | + | | | |
| S. aureus | 2 | − | + | | | T |
| S. auricularis | 1 | − | + | | | |
| S. epidermidis | 2 | − | + | | | |
| S. warneri | 3 | − | + | | | |
| Other bacteria^{d} | 509 | − | − | | | |
| | | VP4 | VP6 | | | |
| V. parahaemolyticus | 43 | + | + | | | |
| V. alginolyticus | 3 | + | + | | | A |
| V. harveyi | 2 | + | + | | | T |
| V. harveyi | 1 | + | + | | | A |
| V. minicus | 1 | + | + | | | T |
| Other bacteria^{d} | 505 | − | − | | | |
| | | LM2 | LM4 | LM5 | LM6 | |
| L. monocytogenes | 40 | + | + | + | + | |
| L. innocua | 2 | + | + | + | + | T |
| L. innocua | 2 | − | − | + | + | T |
| L. ivanovii | 1 | − | − | + | + | |
| L. ivanovii | 1 | + | + | − | − | |
| L. seeligeri | 2 | + | + | − | − | |
| L. welshimeri | 4 | − | + | + | − | |
| L. welshimeri | 2 | − | + | − | + | |
| Other bacteria^{e} | 501 | − | − | − | − | |
| | | SAL | SAL3 | SAL6 | SAL7 | |
| Salmonella spp. | 23 | + | + | + | + | |
| Salmonella spp. | 9 | + | + | + | − | |
| Salmonella spp. | 8 | + | + | − | − | |
| Salmonella spp. | 11 | − | − | + | + | |
| Other bacteria | 504 | − | − | − | − | |
| ^{a}Probe sequences are indicated in Table 2. | | | | | | |
| ^{b}Typical colonies and morphology: B. cereus on MYP agar, pink color, surrounded by precipitate zone; E. coli on L-EMB agar, dark centered, with or without metallic sheen; L. monocytogenes on LPM agar, sparkling blue or white, L. monocytogenes on OXA, colonies with black halo; P. aeruginosa on modified M-PA agar, flat with light outer rims and | | | | | | |
| # brownish to greenish-black centers; Salmonella on BS agar, brown, gray, or black colonies, with or without metallic sheen, Salmonella on HE agar, blue-green to blue with or without black centers, Salmonella on XLD agar, pink with or without black centers; S. aureus on BPA, gray to jet-black, frequently with light-colored margin; and V. parahaemolyticus on TCBS agar, green or blue-green colonies. | | | | | | |

^{c}Typical colony.
^{d}Other bacteria are listed in Table 1.
^{e}Atypical colony.

TABLE 4
Performance of the oligonucleotide array for identification
of seven food and water sanitation microorganisms

| | No. of | | No. of strains | | | |
|---|---|---|---|---|---|---|
| | strains tested | | producing | | | |
| | | Non- | positive reaction | | | |
| | Target | target | | Non- | Sensi- | Spec- |
| | bac- | bac- | Target | target | tivity | ificity |
| Microorganism | teria | teria | bacteria | bacteria | (%) | (%) |
| B. cereus | 26 | 529 | 26 | 6 | 100 | 98.9 |
| E. coli | 48 | 507 | 48 | 0 | 100 | 100 |
| L. monocytogenes | 40 | 515 | 40 | 2 | 100 | 99.6 |
| P. aeruginosa | 29 | 526 | 29 | 0 | 100 | 100 |
| Salmonella spp. | 51 | 504 | 51 | 0 | 100 | 100 |
| S. aureus | 40 | 515 | 38 | 0 | 95 | 100 |
| V. parahaemolyticus | 43 | 512 | 43 | 3 | 100 | 99.4 |

Hybridization of B. cereus to the oligonucleotide array. Of 26 strains of B. cereus tested, all hybridized to probes BC2 and BC4 (Table 3). Two strains of B. mycoides and four strains of B. thuringiensis cross-hybridized to the two probes and produced false-positive reactions. Strains of B. mycoides and B. thuringiensis produced typical colonies (eosing pink, production of lethicinase) as B. cereus on MYP agar (Rhodehamel, E. J., and S. M. Harrnon. 1995.). However, 523 strains (including 18 strains of Bacillus) of other bacteria did not cause cross-hybridization (Table 3). The sensitivity and specificity of the oligonucleotide array for the identification of B. cereus were 100% (26/26) and 98.9% (523/529), respectively (Table 4).

Hybridization of E. coli to the oligonucleotide array: All 48 E. coli strains (including 3 strains of serotype O157:H7) hybridized to probes EC5 and EC7 designed for the species (Table 3). A total of 14 strains belonging to E. fergusonii, Shigella boydii, S. flexneri, and S. sonnei (Table 3) cross-hybridized to probes EC5 and EC7. However, strains of the four cross-hybridizing species could not utilize lactose on L-EMB agar and thus produced a typical colonies (colorless) on the medium. Both sensitivity (48/48) and specificity (507/507) of the oligonucleotide array for the identification of E. coli were 100% (Table 4).

Hybridization of L. monocytogenes to the oligonucleotide array: All 40 strains of L. monocytogenes hybridized to the four probes (LM2, LM4, LM5, and LM6) designed for the identification of the species (Table 3). L. murrayi and L. grayi did not hybridize to any of the four probes, but L. invanovii, L. seeligeri, and L. welshimeri cross-hybridized to two of the four probes. Of the four strains of L. innocua tested, two hybridized to the four probes and produced false-positive reactions; however, the remaining two strains of L. innocua only hybridized to probes LM5 and LM6 (Table 3). L. innocua showed typical colonies on LPM (sparkling blue) and OXA (black halo) agar. The sensitivity and specificity of the oligonucleotide array for the identification of L. monocytogenes were 100% (40/40) and 99.6% (513/515), respectively (Table 4).

Hybridization of P. aeruginosa to the oligonucleotide array: All 29 strains of P. aeruginosa hybridized to probes PA2 and PA6 designed for the species (Table 3). None of the 526 trains of other bacteria including 6 species (13 strains) of Pseudomonas spp. hybridized to probes PA2 and PA6. Both the sensitivity (29/29) and the specificity (526/526) of the oligonucleotide array for the identification of P. aeruginosa were 100% (Table 4).

Hybridization of Salmonella spp. to the oligonucleotide array. Four probes (SAL, SAL3, SAL6, and SAL7) were designed for the identification of Salmonella spp. (Table 2). Twenty-three strains of Salmonella hybridized to the four probes (Table 3). Of the remaining 28 strains of Salmonella, nine hybridized to three probes (SAL, SAL3 and SAL6), eight hybridized to probes SAL and SAL3, and 11 hybridized to probes SAL6 and SAL7 (Table 3). According to the definition of positive reaction of Salmonella, all 51 strains (35 serotypes) of Salmonella were positive by the hybridization assay (Table 3). Both the sensitivity (51/51) and the specificity (504/504) of the oligonucleotide array for the identification of Salmonella were 100% (Table 4).

Hybridization of S. aureus to the oligonucleotide array: Two probes (SA4 and SA5) were used for the identification of S. aureus (Table 2). Of the 40 strains of S. aureus tested, 38 hybridized to both probes (Table 3). Two S. aureus strains only hybridized to probe SA5 but not SA4; therefore, the two strains were false-negatives by the hybridization assay. S. auricularis (1 strain), S. epidermidis (2 strains), and S. warneri (3 strains) cross-hybridized to one (SA5) of the two probes (Table 3). The sensitivity and specificity of the oligonucleotide array for the identification of S. aureus were 95% (38/40) and 100% (515/515), respectively (Table 4).

Hybridization of V. parahaemolyticus to the oligonucleotide array: All 43 strains of V. parahaemolyticus hybridized to probes VP4 and VP6 designed for the species (Table 3). Three strains of V. alginolyticus cross-hybridized to both probes VP4 and VP6, but strains of V. alginolyticus could utilize sucrose (Elliot, E. L. et al. 1995; Farmer, J. J. III et al. 2003) and produced a typical colonies (yellow colonies) on TCBS agar. Three strains of V. harveyi also cross-hybridized to probes VP4 and VP6. Among the three strains of V. harveyi, two were sucrose-negative and produced typical green colonies on TCBS agar, whereas the third one was sucrose-positive (Table 3). In addition, one strain of V. mimicus that was sucrose-negative also hybridized to probes VP4 and VP6. Therefore, three strains (two of V. harveyi and one of V. mimicus) were misidentified as V. parahaemolyticus (Table 3). The sensitivity and specificity of the oligonucleotide array for the identification of V. parahaemolyticus were 100% (43/43) and 99.4% (509/512), respectively (Table 4).

Hybridization of multiple microorganisms to the oligonucleotide array: The present array could be used to simultaneously identify several different target microorganisms grown on their respective selective media. As shown in FIG. 2, the PCR products of two (L. monocytogenes and V. parahaemolyticus), four (B. cereus, E. coli, L. monocytogenes and S. aureus), or six (B. cereus, E. coli, P. aeruginosa, L. monocytogenes, V. parahaemolyticus, and S. aureus) species could concurrently hybridize to their respective probes on the arrays.

Hybridization of microorganisms grown on selective agar to the oligonucleotide array: Ten strains of each of the seven target microorganisms were subcultured on their respective selective media commonly used for their isolation. The hybridization results demonstrated that there were no differences whether the test strains were grown on non-selective or selective media (data not shown).

While embodiments of the present invention have been illustrated described, various modifications and improvements can be made by persons skilled in the art. It is intended that the present invention is not limited to the particular forms as illustrated, and that all the modifications not departing from the spirit and scope of the present invention are within the scope as defined in the appended claims.

What is claimed is:

1. A method for identifying bacteria in a sample which comprises hybridizing an intergenic spacer region separating the 16S and 23S rDNA of the bacteria in the sample with at least one probe selected from the group consisting of BC2 (SEQ ID NO. 1), BC4 (SEQ ID NO. 2), EC5 (SEQ ID NO. 3), EC7 (SEQ ID NO. 4), LM2 (SEQ ID NO. 5), LM4 (SEQ ID NO. 6), LM6 (SEQ ID NO. 7), LM5 (SEQ ID NO. 8), PA2 (SEQ ID NO. 9), PA6 (SEQ ID NO. 10), SAL (SEQ ID NO. 11), SAL3 (SEQ ID NO. 12), SAL6 (SEQ ID NO. 13), SAL7 (SEQ ID NO. 14), SA4 (SEQ ID NO. 15), SA5 (SEQ ID NO. 16), VP4 (SEQ ID NO. 17), VP6 (SEQ ID NO. 18), the complement thereof, and the variant thereof.

2. The method according to claim 1, wherein
if the intergenic spacer region is hybridized with BC2, BC4, the complement thereof, or the variant thereof, the bacterium is identified as Bacillus cereus;
if the intergenic spacer region is hybridized with EC5, EC7, the complement thereof, or the variant thereof, the bacterium is identified as Escherichia coli;
if the intergenic spacer region is hybridized with LM2, LM4, LM6, LM5, the complement thereof, or the variant thereof, bacterium is are identified as Listeria monocytogenes;
if the intergenic spacer region is hybridized with PA2, PA6, the complement thereof, or the variant thereof, the bacterium is identified as Pseudomonas aeruginosa;
if the intergenic spacer region is hybridized with two or more probes of SAL, SAL3, SAL6, SAL7, the complement thereof, or the variant thereof, the bacterium is identified as Salmonella spp.;
if the intergenic spacer region is hybridized with SA4, SA5, the complement thereof, or the variant thereof, the bacterium is identified as Staphylococcus aureus; and
if the intergenic spacer region is hybridized with VP4, VP6, the complement thereof, or the variant thereof, the bacterium is identified as Vibrio parahaemolyticus.

3. The method according to claim 1, wherein the sample is food or water.

4. The method according to claim 1, wherein prior to the hybridization, the intergenic spacer region separating the 16S and 23S rDNA of the bacteria in the sample is amplified in a polymerase chain reaction.

5. The method according to claim 4, wherein the intergenic spacer region separating the 16S and 23S rDNA of the bacteria in the sample is amplified with bacteria-specific universal primers.

6. The method according to claim 5, wherein the bacteria-specific universal primers comprise 2F primer (SEQ ID NO. 21) and 6R primer (SEQ ID NO. 22).

7. The method according to claim 6, wherein the amplified intergenic spacer region is hybridized with the probes comprising BC2, BC4, EC5, EC7, LM2, LM4, LM6, LM5, PA2, PA6, SAL, SAL3, SAL6, SAL7, SA4, SA5, VP4, VP6, the complement thereof, and the variant thereof.

8. The method according to claim 1, wherein the probe(s) is (are) coated on a substrate.

9. The method according to claim 1, wherein the hybridization is carried out by using a microarray.

10. The method according to claim 1 further comprising a positive control step which comprises hybridizing the intergenic spacer region separating the 16S and 23S rDNA of Xanthobacter flavus with at least one probe of PC1 (SEQ ID NO. 19) and PC2 (SEQ ID NO. 20).

11. A kit for identifying bacteria in a sample comprising at least one probe selected from the group consisting of BC2 (SEQ ID NO. 1), BC4 (SEQ ID NO. 2), EC5 (SEQ ID NO. 3), EC7 (SEQ ID NO. 4), LM2 (SEQ ID NO. 5), LM4 (SEQ ID NO. 6), LM6 (SEQ ID NO. 7), LM5 (SEQ ID NO. 8), PA2 (SEQ ID NO. 9), PA6 (SEQ ID NO. 10), SAL (SEQ ID NO. 11), SAL3 (SEQ ID NO. 12), SAL6 (SEQ ID NO. 13), SAL7 (SEQ ID NO. 14), SA4 (SEQ ID NO. 15), SA5 (SEQ ID NO. 16), VP4 (SEQ ID NO. 17), VP6 (SEQ ID NO. 18), the complement thereof, and the variant thereof.

12. The kit according to claim 11, wherein the sample is food or water.

13. The kit according to claim 11 further comprising reagents for obtaining the intergenic spacer region separating the 16S and 23S rDNA of the bacteria.

14. The kit according to claim 13, wherein the reagents are for polymerase chain reaction.

15. The kit according to claim 14 further comprising bacteria-specific universal primers.

16. The kit according to claim 15, wherein the bacteria-specific universal primers comprise 2F primer (SEQ ID NO. 21) and 6R primer (SEQ ID NO. 22).

17. The kit according to claim 16, wherein the probes comprise BC2, BC4, EC5, EC7, LM2, LM4, LM6, LM5, PA2, PA6, SAL, SAL3, SAL6, SAL7, SA4, SA5, VP4, VP6, the complement thereof, and the variant thereof.

18. The kit according to claim 11 further comprising reagents for hybridization.

19. The kit according to claim 11, wherein the probe(s) is (are) coated on a substrate.

20. The kit according to claim 18 further comprising reagents for hybridization using a microarray.

21. The kit according to claim 11 further comprising an intergenic spacer region separating the 16S and 23S rDNA of Xanthobacter flavus and at least one probe of PC1 (SEQ ID NO. 19) and PC2 (SEQ ID NO. 20).
